(19) United States
(12) Patent Application Publication
(10) Pub. No.: US 2014/0273008 A1
(43) Pub. Date: Sep. 18, 2014

(54) METHODS TO ASSESS ENZYME ACTIVITY USING MASS SPECTROMETRIC IMMUNOASSAY

(71) Applicants: Urban A. Kiernan, Chandler, AZ (US); Eric E. Niederkofler, Phoenix, AZ (US)

(72) Inventors: Urban A. Kiernan, Chandler, AZ (US); Eric E. Niederkofler, Phoenix, AZ (US)

(73) Assignee: Molecular BioProducts, Inc., San Diego, CA (US)

(21) Appl. No.: 14/208,519

(22) Filed: Mar. 13, 2014

Related U.S. Application Data
(60) Provisional application No. 61/788,290, filed on Mar. 15, 2013.

(51) Int. Cl. G01N 33/573

(52) U.S. Cl. CPC G01N 33/573
USPC 435/7.4

(57) ABSTRACT

Methods to assess biological enzyme activity directly in a complex clinical sample. To such a sample, e.g., plasma, an exogenous synthetic peptide substrate for an enzyme present in the sample and to be assessed is added. The sample then undergoes mass spectrometric immunoassay (MSIA) to monitor the mass spectral profile of the immuno-purified exogenous synthetic peptide substrate directly from the sample. Using the resulting profile, parameters such as enzyme activity and effect of enzyme modulators, including therapeutics administered to an individual for a condition involving the enzyme, may be determined.

This application claims priority from U.S. Application Ser. No. 61/788,290 filed Mar. 15, 2013, which is expressly incorporated by reference herein in its entirety.

A method of determining enzyme activity using mass spectrometric immunoassay (MSIA) is disclosed. The method determined enzyme activity as related to, e.g., disease determination, drug development, therapeutic treatment, etc. In one embodiment, the method was used to assess activity of endogenous enzymes and/or enzyme systems or cascades that are targets for therapy to ameliorate pathologies, or symptoms or sequela of pathologies. In one embodiment, the MSIA method determined activity of the endogenous exopeptidase enzyme diphenyl peptidase-4 (DPP4), a class of enzymes implicated in indications such as diabetes mellitus, and the method was used to assess efficacy of various inhibitors of DPP4.

The disclosed methods used MSIA to affinity-retrieve the reacted and unreacted substrate for specific enzymes, such as DPP4, to be analyzed from a complex biological sample. The monitored conversion of this exogenous synthetic peptide substrate to product is in direct relation to the activity of the functional enzyme present in a patient. For the enzyme whose activity is to be determined, an artificial peptide was introduced into the clinical sample in vitro. The mass spectral measurement of this peptide and its resultant product provided a metric of enzyme activity within the patient, with the specific exogenous synthetic peptide substrate as a proxy to measure activity of native enzyme in the patient. MS quantitation of the ratio of unreacted (i.e., not enzyme cleaved) specific exogenous synthetic peptide substrate: reacted (enzyme cleaved) synthetic substrate was used to determine enzyme activity in the clinical sample, and hence assessed enzyme activity in the individual from whom the sample was obtained.

One embodiment of the method is a mass spectrometric immunoassay (MSIA) to assess activity of any native enzyme, human or animal, in vitro as an indication of its activity in vivo. In this embodiment, a specific exogenous synthetic peptide substrate for a native enzyme is added to the clinical sample containing the enzyme, the specific exogenous synthetic peptide substrate being a proxy for activity of the native enzyme. MS analysis quantitates the ratio of the immunoaffinity purified unreacted exogenous synthetic peptide substrate to reacted products, if any, resulting from the enzyme's activity on the exogenous synthetic peptide substrate. The enzyme's activity is assessed using the ratio, where a greater difference indicates relatively higher enzyme activity.

This MSIA method is useful for any or all of the following assessments: correlating substrate decrease with product increase for quality control, determining efficacy of therapy affecting a particular enzyme, determining compliance with therapy affecting a particular enzyme, determining enzyme pharmacokinetics e.g., EC50, ID50, etc., determining potential enzyme inhibitors and evaluating their efficacy, determining inhibitor or potential inhibitor synergy, etc. In one embodiment, the method is used to create a standard activity curve for specific activity determination of any enzyme incubated under any desired reaction condition, e.g., elevated temperature, increased time, etc. In one embodiment, the method is used for patient stratification based on enzyme activity.

### BRIEF DESCRIPTION OF THE DRAWINGS

FIG. 1 illustrates a general mass spectrometry immunoassay (MSIA) workflow for assessing enzyme activity.

FIG. 2 shows results of one substrate conversion assessment in human plasma using MSIA.

FIG. 3 shows results of another substrate conversion assessment in human plasma using MSIA.

FIG. 4 shows results of a substrate conversion assessment in human plasma using MSIA in the presence of an enzyme inhibitor.

FIG. 5 shows results of a substrate conversion assessment in human plasma using MSIA in the presence of a different enzyme inhibitor.

FIGS. 6A-6B show a representative mass spectrum (FIG. 6A) and results of a substrate conversion (FIG. 6B) using a specific exogenous synthetic peptide substrate.

MSIA is a method by which sample purification and preparation occurs essentially simultaneously with sample aliquot and delivery. More specifically, a pipette tip, into which a specific volume of a clinical sample, human or animal, is drawn, contains an affinity reagent for selective preparation and immuno-affinity purification of the sample for subsequent direct mass spectrometer analysis; i.e., no intervening purification steps are performed. As shown schematically in FIG. 1, a clinical sample is drawn into a pipette tip containing an exogenous synthetic peptide substrate. The clinical sample and the exogenous peptide substrate are incubated to effect binding of the exogenous peptide substrate to a specific enzyme that is present in the clinical sample. After washes with buffer and water, the eluant is used for subsequent MS analysis. In this elution process, the eluate is dispensed into a microtiter plate for LC/MS application, directly into a LC/MS, or onto a target for MS insertion. Regardless of the MS technique applied, the eluant contained the exogenous peptide substrates, both the parent exogenous peptide substrate and the cleaved degraded product that was produced based on the activity of the enzyme contained in the clinical sample.

In one embodiment, the enzyme assessed was diphenyl peptidase-4 (DPP4). DPP4 is an antigenic exopeptidase. DPP4 cleaves amino acids from the N-terminus at a specific sequence, namely, the N-terminal of X-proline or X-alanine, thus there are a limited number of proteins on which it will react compared to some enzymes, e.g., trypsin, that react with any arginine anywhere in the protein or polypeptide.

DPP4 is a target in the study and treatment of diabetes because it is responsible for the degradation of glucagon-like peptide 1 (GLP1) and other secretins. DPP4 activity is also influential in the degradation and deactivation of other hormone and signaling peptides associated with other indications. There is a class of anti-type 2 diabetes mellitus therapeutics that specifically target DPP4 for inhibition. These inhibitors, also known as gliptins, have recently been under scrutiny by the U.S. Food and Drug Administration (FDA) for newly observed safety concerns.

In one embodiment, the disclosed methods determined the activity of the endogenous enzyme DPP4, and also assessed DPP4 inhibitor effectiveness using MSIA. More specifically, the disclosed methods introduced an exogenous synthetic peptide substrate having a cleavage site for DPP4 into an in vitro patient sample, where the patient sample contained the enzyme, and used MSIA to monitor enzyme activity, i.e., cleavage, of the N-terminal X-proline or X-alanine amino acids in the synthetic peptide substrate. The mass spectral measurement of the exogenous synthetic peptide substrate provided a metric of DPP4 activity within the patient. Assessments such as these have applications in specific areas of biopharma and clinical diagnostics in which DPP4 activity is of critical importance and growing value due to the development of novel therapeutic compounds, e.g., gliptins and secretin mimetics, that are used in the treatment of type 2 diabetes mellitus. The disclosed methods have utility in, e.g., compound screening, patient selection, dosing range finding, and assessing treatment efficacy.

The specific exogenous synthetic peptide substrate for a particular enzyme may be designed with, but not limited to, specific amino acid sequences, specific glycol-residues, and/or functional groups such as phosphates. In only one embodiment of the method, the specific exogenous synthetic peptide substrate for DPP4 was recombinant GLP1, but the method is not so limited and any peptide containing the cleavage site for DPP4 may be used.

Commercially available DPP4 inhibitor screening kits contain reagents (e.g., a recombinant DPP4 source, buffers, and substrate), for use in compound screening and establishing the kinetic properties of such compounds. These same reagents are often used in establishing endogenous DPP4 activity, using either human plasma or cell culture. These assays are optically based, and many are fluorescent. One of the problems with optically based analysis of such complex matrices is signal inconsistencies due to heavy background. This background stems from within the purity of the sample being analyzed. The complexity of the DPP4 source matrix contains interfering substances such as particulates and naturally fluorescent materials that influence optical measurements. Measurements on such a matrix are problematic and require large dilutions in order to obtain somewhat consistent results.

The disclosed method used MSIA in the absence of optical activity assay method, and thus improves over such commercially available DPP4 inhibitor screening kits. In the disclosed method, the substrate to be analyzed is affinity retrieved from the complex biological media. This purification process in the analytical workflow provided more consistent and reproducible results. Such methods have great impact on the in vitro evaluation of enzymes such as DPP4 from patients, creating commercial potential in various aspects of both research and development and clinical fields.

An exogenous synthetic peptide that is a substrate for the enzyme to be assessed is introduced into a clinical specimen, e.g., blood, urine, plasma, cerebrospinal fluid, lymphatic fluid, etc., i.e., the clinical sample is spiked or doped with the exogenous synthetic peptide substrate. For example, if the enzyme to be assessed is DPP4, the sample may be spiked with an exogenous peptide substrate for DPP4, e.g., recombinant GLP1, natriuretic peptide, etc. The exogenous synthetic peptide substrate that is added to the clinical sample in vitro is termed the parent substrate. The sample is then analyzed by MS, or is incubated for a set period of time before MS analysis. Incubation may be for any desired time period, and at any desired temperature, e.g., ambient temperature 19° C.-22° C., or at another desirable temperature, e.g., 37° C. Enzyme that is present in the clinical sample proteolytically cleaves the exogenous parent synthetic peptide substrate into a smaller fragment. The exogenous synthetic peptide substrates, both the parent (unreacted) and the cleaved (reacted) degraded product, are the targets that are monitored by subsequent MSIA. The method may be summarized as follows:

(a) adding an exogenous synthetic peptide that is a substrate for an enzyme to be assessed to a clinical sample containing that enzyme in vitro,

(b) thereafter obtaining a mass spectral measurement of the exogenous synthetic peptide substrate in the sample using MSIA, and

(c) using the mass spectral measurement of sample to assess at least one parameter indicating the activity of the enzyme in the sample.

Incubation occurs after step (a) and before step (b) or continuing after step (b) if a time course analysis is performed.

In one embodiment, a MS measurement of the exogenous synthetic peptide substrate in the sample (step (b)) is performed at a single time point, e.g., 15 min. In one embodiment, a MS measurement of the exogenous synthetic peptide substrate in the sample (step (b)) is performed over a time course, e.g., from 100% substrate to decreased or essentially depleted substrate. The time course may be performed utilizing sequential aliquot removal from the same clinical sample, or may be performed on multiple samples assayed in parallel. In all embodiments, the clinical samples are analyzed using MSIA, which selectively targets the substrate peptide, both reacted and non-reacted. MS detection allows for differentiation of the two forms, i.e., the reacted (cleaved, degraded) form of the substrate peptide, and unreacted parent form of the substrate peptide. It thus provides a method of analyzing the activity of an enzyme in a biological system.

Any MS system may be used with the disclosed method including, but not limited to, MALDI or ESI ionization techniques that are used with time of flight, triple quad, iontrap or orbi trap mass spectrometers.

It will be appreciated that the following examples are not limiting and that the method is useful with other human and non-human clinical samples, other incubation times, single aliquot analysis, other temperatures, other MS detection systems, etc.

As one example FIG. 2, DPP4 Activity—Human Plasma 1, shows observed substrate conversion in a human heparinized plasma clinical sample that was spiked with a recombinant DPP4 substrate peptide. In FIG. 2, the abundance plot shows detected substrate (line starting at 100% of total substrate) and DPP4 reacted products (line starting at 0% of total substrate) over a 75 min time course. The following results were obtained:

| Time (min) | % Substrate | % Product |
|---|---|---|
| 0 | 100 | 0 |
| 15 | 40.7 | 59.3 |
| 30 | 18.5 | 81.5 |
| 45 | 12.9 | 87.1 |
| 60 | 7.2 | 92.8 |
| 75 | 5.9 | 94.1 |

The results demonstrated that the exogenous synthetic peptide substrate was proteolytically degraded by DPP4 within the 75 minute time course study. The plotted rate of change over this time course was directly proportional to the functional activity of DPP4 found in the sample and, therefore, within the individual. Such data are not normally or easily generated, and are valuable as a bioassay for a number of clinical and research and development purposes.

As another example FIG. 3, DPP4 Activity—Human Plasma 2, shows observed substrate conversion in a human heparinized plasma clinical sample that was spiked with a recombinant DPP4 substrate peptide. In FIG. 3, the abundance plot shows detected substrate (line starting at 100% of total substrate) and DPP4 reacted products (line starting at 0% of total substrate) over a 75 min time course. The following results were obtained:

| Time (min) | % Substrate | % Product |
|---|---|---|
| 0 | 100 | 0 |
| 15 | 80.9 | 19.1 |
| 30 | 62.2 | 37.8 |
| 45 | 54.8 | 45.2 |
| 60 | 47.2 | 52.8 |
| 75 | 39 | 61 |

The results demonstrated that the exogenous synthetic peptide substrate peptide was proteolytically degraded by DPP4 within the 75 minute time course study. The plotted rate of change over this time course was directly proportional to the functional activity of DPP4 found in the sample and, therefore, within the individual.

In embodiments, clinical samples were treated with various inhibitors to determine the modulation, if any, of the endogenous enzyme activity. For example, if DPP4 was the enzyme whose activity was to be assessed, inhibitors were used that were known or speculated to inhibit DPP4 activity. Concentration studies were also performed (data not shown). The results from inhibitor treatment studies are shown in FIGS. 4 and 5.

As one example using the inhibitor leupeptin, FIG. 4 DPP4 Activity—Human Plasma 1 with 1 mM Leupeptin, shows observed substrate conversion in a human heparinized plasma clinical sample that was spiked with a recombinant DPP4 substrate peptide and 1 mM leupeptin. In FIG. 4, the abundance plot shows detected substrate (line starting at 100% of total substrate) and DPP4 reacted products (line starting at 0% of total substrate) over a 75 min time course. The following results were obtained with the leupeptin inhibitor:

| Time (min) | % Substrate | % Product |
|---|---|---|
| 0 | 100 | 0 |
| 15 | 47.4 | 52.6 |
| 30 | 22.2 | 77.8 |
| 45 | 14.9 | 85.1 |
| 60 | 12.1 | 87.9 |
| 75 | 9.8 | 90.2 |

The results of this inhibition experiment showed that the substrate peptide was still proteolytically converted during the time course study.

As another example using the inhibitor AEBSF, FIG. 5 DPP4 Activity—Human Plasma 1 with 5 mM AEBSF, shows observed substrate conversion in a human heparinized plasma clinical sample that was spiked with a recombinant DPP4 substrate peptide and 5 mM AEBSF. In FIG. 5, the abundance plot shows detected substrate (line starting at 100% of total substrate) and DPP4 reacted products (line starting at 0% of total substrate) over a 75 min time course. The following results were obtained with the AEBSF inhibitor:

| Time (min) | % Substrate | % Product |
|---|---|---|
| 0 | 100 | 0 |
| 15 | 88.3 | 11.7 |
| 30 | 82.6 | 17.4 |
| 45 | 77.8 | 22.2 |
| 60 | 75.7 | 24.3 |
| 75 | 75.2 | 24.8 |

FIG. 6 shows the observed substrate conversion by the enzyme DPP4 in a human heparinized plasma sample spiked with the exogenous synthetic peptide substrate glucagon-like peptide-1 (7-36) amide (GLP1 (7-36A); MW=3297.6). The MS generated was done using MALDI-TOF MS. FIG. 6A shows representative mass spectra: the two MS traces shown in FIG. 6A are the 0 (upper trace) and the 75 minute (lower trace) time points. At 75 minutes nearly all the GLP1(7-36A) had been digested to GLP1(9-36A); MW=3090.4. FIG. 6B shows the percentage data plot of the substrate conversion at 15 minute time points over the 75 minute time course.

The inventive system and method used an exogenous synthetic peptide substrate whose conversion to product, using enzyme present in a clinical sample, human or animal, to assess kinetic activity of an endogenous enzyme. The inventive system and method may be used in bioassays for enzyme kinetic studies in the determination of catalytic reagents. An enzyme of any classification may be monitored by the method: oxidoreductases, transferases, hydrolases, lyases, isomerases, and/or ligases. In one embodiment, the enzyme monitored is a protease, also known as a peptidase, that catalytically hydrolyzes or cleaves certain peptide bonds in a protein. In one embodiment, the exopeptidase DPP4 was monitored. These data demonstrated the utility and capability of MSIA for monitoring and assessing enzyme activity in a complex biological sample such as plasma without the need for sample preparation and/or dilution and without a lengthy kinetic study which can take over 48 hours. MSIA was used to immuno-affinity isolate, then mass spectrometrically analyze, the unreacted (parent) and/or reacted synthetic peptide substrate. The complex clinical sample evaluated was artificially modulated by induction of various enzyme inhibitors. The presence of these inhibitors directly influenced the rate of degradation of the exogenous synthetic peptide substrate, which was then monitored. The selective enrichment and purification of the exogenous synthetic peptide substrate, both unreacted (undegraded) and reacted (degraded) provided an advantageous unique approach to the routine monitoring of enzyme function.

Although the invention has been described in detail for the purposes of illustration, it is understood that such detail is solely for that purpose, and variations can be made therein by those skilled in the art without departing from the spirit and scope of the claimed invention.

What is claimed is:

1. A method for biological enzyme activity assessment directly in a clinical sample, the method comprising the steps of
(a) adding to a clinical sample in vitro an exogenous synthetic peptide substrate for an enzyme to be assessed in the clinical sample,
(b) performing mass spectrometric immunoassay (MSIA) to obtain a mass spectral measurement of a purified exogenous synthetic peptide substrate directly in the sample, and
(c) using the mass spectral measurement of the purified exogenous synthetic peptide substrate to obtain at least one parameter indicating the activity of the enzyme in the sample, assessing biological enzyme activity directly in a clinical sample.

2. The method of claim 1 further comprising quantitating the exogenous synthetic peptide substrate and reacted products, if any, resulting from enzyme activity on the exogenous synthetic peptide substrate in step (b).

3. The method of claim 2 further comprising determining a ratio of quantity of unreacted exogenous synthetic peptide substrate: quantity of reacted exogenous synthetic peptide substrate in step (c) to determine enzyme activity wherein a relatively greater difference between unreacted: reacted indicates relatively higher enzyme activity.

4. The method of claim 1 further comprising incubating the sample after step (a) and before step (b).

5. The method of claim 4 wherein the incubation is at room temperature.

6. The method of claim 1 wherein step (b) is performed at a single time point.

7. The method of claim 1 wherein step (b) is performed at a plurality of time points.

8. The method of claim 7 wherein step (b) is performed using sequential aliquot removal from a single clinical sample.

9. The method of claim 7 wherein step (b) is performed using single aliquot removal from a plurality of samples in parallel.

10. The method of claim 1 wherein the enzyme is diphenyl peptidase-4 (DPP4).

11. The method of claim 1 wherein the clinical sample is selected from the group consisting of blood, plasma, serum, urine, cerebrospinal fluid, lymphatic fluid, and combinations thereof.

12. A mass spectrometric immunoassay (MSIA) method to measure activity of a native enzyme in vivo, the method comprising
adding a specific exogenous synthetic peptide substrate for a native enzyme to a clinical sample containing or thought to contain the enzyme, the specific exogenous synthetic peptide substrate being a proxy for activity of the native enzyme,
using mass spectrometry (MS) to quantitate a ratio of the immuno-affinity purified exogenous synthetic peptide substrate and reacted products, if any, resulting from the enzyme activity on the exogenous synthetic peptide substrate, and
determining the enzyme activity based on the ratio wherein a greater difference indicates relatively higher enzyme activity, measuring activity of a native enzyme in vivo.

13. The method of claim 12 wherein the enzyme activity is determined for a purpose selected from the group consisting of correlating substrate decrease with product increase for quality control, determining efficacy of therapy affecting an enzyme, determining compliance with therapy affecting an enzyme, determining enzyme kinetics, determining inhibitor effect on enzyme kinetics, determining activator effect on enzyme kinetics, and combinations thereof.

14. The method of claim 12 performed under conditions to generate a standard curve for activity of the enzyme.

15. The method of claim 12 wherein the enzyme is diphenyl peptidase-4.

16. A method to assess diphenyl peptidase-4 (DPP4) activity in a patient clinical sample, the method comprising the steps of
(a) adding to a patient clinical sample in vitro an exogenous synthetic peptide substrate containing a cleavage site for DPP4,
(b) performing mass spectrometric immunoassay (MSIA) to obtain a mass spectral measurement of the purified exogenous synthetic peptide substrate directly in the sample over a time course,
(c) using the mass spectral measurement to quantitate the unreacted exogenous synthetic peptide substrate compared to the reacted exogenous synthetic peptide substrate over the time course, and
(d) assessing DPP4 activity by the comparison, where a relatively higher reacted to unreacted quantity indicates relatively higher enzyme activity in the clinical sample, assessing DPP4 activity in a patient clinical sample.

17. The method of claim 16 wherein the clinical sample is from a patient with diabetes mellitus.

18. The method of claim 16 wherein the clinical sample is from a patient receiving gliptin therapy for diabetes mellitus.
